(19) United States
(12) Patent Application Publication
(10) Pub. No.: US 2025/0368901 A1
(43) Pub. Date: Dec. 4, 2025

(54) METHODS OF MAKING LIGHT OLEFINS THAT INCLUDE MODIFYING CATALYSTS

(71) Applicant: Dow Global Technologies LLC, Midland, MI (US)

(72) Inventors: Manish Sharma, Freeport, TX (US); Brian W. Goodfellow, Freeport, TX (US); Lin Luo, Freeport, TX (US); Andrzej Malek, Midland, MI (US)

(73) Assignee: Dow Global Technologies LLC, Midland, MI (US)

(21) Appl. No.: 18/873,942

(22) PCT Filed: Jun. 9, 2023
(86) PCT No.: PCT/US2023/068192

§ 371 Date: Dec. 11, 2024

Related U.S. Application Data
(60) Provisional application No. 63/352,020, filed on Jun. 14, 2022.

(51) Int. Cl. C10G 29/04; B01J 21/04; B01J 21/08; B01J 23/08; B01J 23/34; B01J 23/42; B01J 23/745; B01J 37/02

(52) U.S. Cl. CPC C10G 29/04; B01J 21/04; B01J 21/08; B01J 23/08; B01J 23/34; B01J 23/42; B01J 23/745; B01J 37/02; C10G 2400/20

(57) ABSTRACT

A method may include operating a dehydrogenation process whereby a hydrocarbon-containing feed is converted to light olefins, wherein the dehydrogenation process utilizes a fluidized process catalyst that circulates between a reactor and a combustor. The method may comprise withdrawing the process catalyst from the dehydrogenation process, modifying the process catalyst to form a modified catalyst, and adding the modified catalyst back to the dehydrogenation process. The process catalyst may include from 0.1 wt. % to 10 wt. % of one or more metals chosen from gallium, indium, thallium, or combinations thereof, from 1 ppmw to 1000 ppmw of one or more metals chosen from platinum, palladium, rhodium, iridium, ruthenium, osmium, or combinations thereof, and at least 85 wt. % support. Modifying the process catalyst may include adding one or more of manganese, iron, chromium, or vanadium.

### CROSS REFERENCE TO RELATED APPLICATION

This application claims priority to U.S. Provisional Application Ser. No. 63/352,020, filed Jun. 14, 2022, which is hereby incorporated by reference in its entirety.

### TECHNICAL FIELD

Embodiments described herein generally relate to chemical processing and, more specifically, to methods and systems for light olefin production.

### BACKGROUND

Light olefins, such as propylene, may be used as base materials to produce many different materials, such as polypropylene, isopropanol, and acrylic acid, which may be used in, e.g., packaging, construction, and textiles. As a result of this utility, there is a worldwide demand for light olefins. Suitable processes for producing light olefins generally depend on the given chemical feed and include those that utilize fluidized catalysts. For example, light olefins may be formed by the catalytic dehydrogenation of alkanes in a fluidized bed reactor. However, there is a need for improvement in the systems and associated catalysts used to make light olefins.

### SUMMARY

Some methods and associated systems used to make light olefins may utilize a catalyst which may be cycled between a reactor, where light olefins are produced in an endothermic reaction, and a combustor, where the catalyst is heated by exothermic combustion of at least a supplemental fuel (sometimes along with combustion of coke). Such catalysts may have catalytic activity not only for the dehydrogenation of alkanes, but also for the combustion of supplemental fuels. Some embodiments of such suitable catalysts include, e.g., gallium and platinum on a support. In some embodiments, conventional catalysts used for dehydrogenation may over a period of use, suffer from lowered catalytic activity, as compared to a conventional catalyst that has not yet been used in a dehydrogenation process. Such used catalysts may no longer sufficiently catalyze dehydrogenation of alkanes, the combustion of supplemental fuels, or both. As is described herein, it has been discovered that a method of modifying partially deactivated catalysts by adding metals such as manganese, iron, chromium, or vanadium may restore catalytic activity for the dehydrogenation of alkanes, combustion activity, or both as compared with conventional catalysts that, for example, have not been modified by adding metals such as manganese, iron, chromium, or vanadium.

According to one or more embodiments of the present disclosure, a method may comprise operating a dehydrogenation process whereby a hydrocarbon-containing feed is converted to light olefins, wherein the dehydrogenation process utilizes a fluidized process catalyst that circulates between a reactor and a combustor. The method may further comprise withdrawing the process catalyst from the dehydrogenation process, modifying the process catalyst to form a modified catalyst, and adding the modified catalyst back to the dehydrogenation process. Where the process catalyst comprises from 0.1 wt. % to 10 wt. % of one or more metals chosen from gallium, indium, thallium, or combinations thereof, from 1 ppmw to 1000 ppmw of one or more metals chosen from platinum, palladium, rhodium, iridium, ruthenium, osmium, or combinations thereof, and at least 85 wt. % support. Modifying the process catalyst may comprise adding one or more of manganese, iron, chromium, vanadium, or aluminum.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawing and claims, or recognized by practicing the described embodiments. The drawing is included to provide a further understanding of the embodiments and, together with the detailed description, serves to explain the principles and operations of the claimed subject matter. However, the embodiment depicted in the drawing is illustrative and exemplary in nature, and not intended to limit the claimed subject matter.

### BRIEF DESCRIPTION OF THE DRAWINGS

The following detailed description may be better understood when read in conjunction with the following drawing, in which:

FIG. 1 depicts a flow chart that includes steps for making light olefins, according to one or more embodiments of the present disclosure; and

FIG. 2 schematically depicts a reactor system, according to one or more embodiments of the present disclosure.

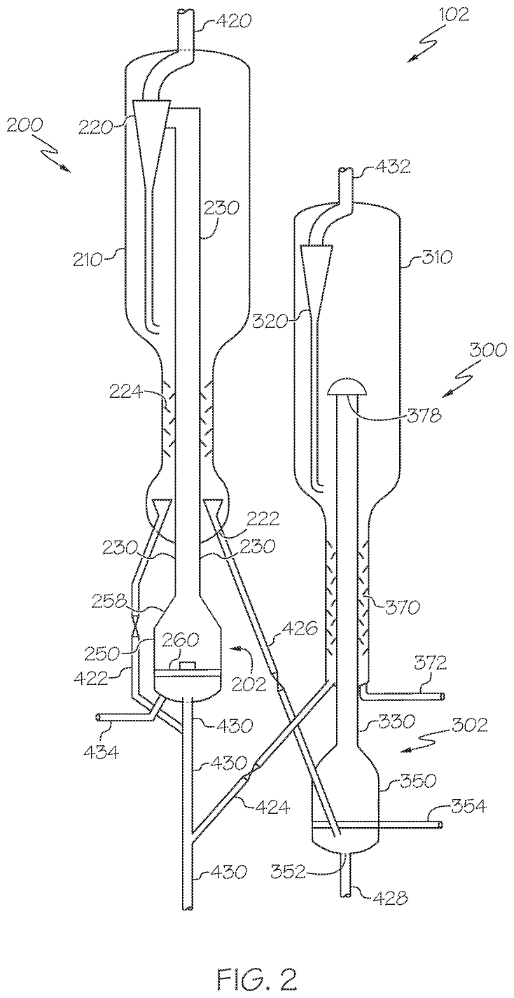

When describing the simplified schematic illustration of FIG. 2, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

Reference will now be made in greater detail to various embodiments, some of which are illustrated in the accompanying drawing.

### DETAILED DESCRIPTION

The present disclosure is directed to methods for making light olefins by dehydrogenation which may include steps such as operating a dehydrogenation process, withdrawing a process catalyst from the dehydrogenation process, modifying the process catalyst to form a modified catalyst, and adding the modified catalyst back to the dehydrogenation process. The process catalyst may comprise from 0.1 wt. % to 10 wt. % of one or more metals chosen from gallium, indium, thallium, or combinations thereof, from 1 to 1000 ppmw of one or more metals chosen from platinum, palladium, rhodium, iridium, ruthenium, osmium, or combinations thereof, and at least 85 wt. % support. As described herein, the modifying of the process catalyst may include the adding of one or more of manganese, iron, chromium, or vanadium. In one or more embodiments, such modified catalysts provide improved dual catalytic functionality for dehydrogenation of alkanes as well as for combustion of supplemental fuels. Such modified catalysts may be particularly well suited for fluidized dehydrogenation of light alkanes to light olefins.

As is described herein, the modification of the process catalyst to the modified catalyst, and its subsequent use in dehydrogenation processes, may allow for reusing/recycling continued use of the process catalyst, which still may contain valuable materials such as platinum and gallium. However, according to one or more embodiments, the addition of manganese, iron, chromium, or vanadium may enhance dehydrogenation activity, combustion activity, or both.

As used in the present disclosure, the term “process catalyst” refers to a catalyst that has been used in a dehydrogenation process. A process catalyst may have a composition different than that of a fresh catalyst that has not yet been introduced to a dehydrogenation process. The modified catalyst is a process catalyst which has undergone modification as described herein, and is generally re-inserted into the dehydrogenation process.

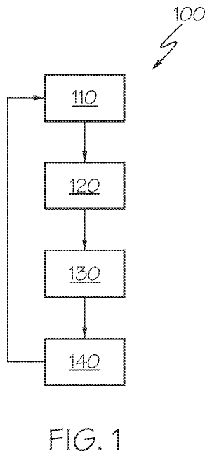

Referring to FIG. 1, the steps of a method for making light olefins 100 is shown, according to one or more embodiments described herein. FIG. 1 depicts, in sequential order, step 110 of operating a dehydrogenation process, step 120 of withdrawing the process catalyst, step 130 of modifying the process catalyst to form a modified catalyst, and step 140 of loading the modified catalyst back into the dehydrogenation process.

Step 110 generally includes operating a dehydrogenation process. In the operating of the dehydrogenation process in step 110, a hydrocarbon-containing feed is converted to light olefins in an olefin-containing effluent. The dehydrogenation process of step 110 may be performed using a reactor system as schematically depicted in FIG. 2. The steps of FIG. 1 are sometimes described in the context of the reactor system of FIG. 2, as described herein. It should be understood that when describing the systems and associated methods of FIG. 2, the described “catalyst” may refer to the process catalyst or the modified catalyst, unless specified. Generally, the movement and fluidization of the process catalyst and the modified catalyst through the system of FIG. 2 is identical.

Embodiments of the methods presently disclosed will now be described in detail herein in the context of the reactor system of FIG. 2 operating as a fluidized dehydrogenation reactor system to produce light olefins, such as propylene. However, it should be understood that the principles disclosed and taught herein may be applicable to other systems which utilize different system components oriented in different ways. For example, the concepts described herein may be equally applied to other systems with alternate reactor units and regeneration units, such as those that operate under non-fluidized conditions or include downers rather than risers. It should be further understood that not all portions of FIG. 2 should be construed as essential to the claimed subject matter. Moreover, while the recited method steps in the appended claims are described herein in the context of FIG. 2, such recited method steps should be understood as adaptable to other systems, as would be understood by those skilled in the art.

Now referring to FIG. 2, an example reactor system 102 that may be suitable for use with the methods and/or apparatuses described herein is schematically depicted. The reactor system 102 generally comprises multiple system components, such as a reactor portion 200 and a catalyst processing portion 300. As described herein, “system components” refer to portions of the reactor system 102, such as reactors, separators, transfer lines, combinations thereof, and the like. As used herein in the context of FIG. 2, the reactor portion 200 generally refers to the portion of the reactor system 102 in which the major process reaction takes place (e.g., dehydrogenation) to form the olefin-containing effluent. A hydrocarbon-containing feed enters the reactor portion 200, is contacted with a catalyst, converted to an olefin-containing effluent (containing product and unreacted feed), and exits the reactor portion 200. The reactor portion 200 comprises a reactor 202 which may include an upstream reactor section 250 and a downstream reactor section 230. According to one or more embodiments, as depicted in FIG. 2, the reactor portion 200 may additionally include a catalyst separation section 210, which serves to separate the catalyst from the olefin-containing effluent formed in the reactor 202. Also, as used herein, the catalyst processing portion 300 generally refers to the portion of the reactor system 102 where the catalyst is in some way processed, such as by combustion, to, e.g., improve catalytic activity by decoking and/or heating the catalyst. The catalyst processing portion 300 may comprise a combustor 350 and a riser 330, and may additionally comprise a catalyst separation section 310. In one or more embodiments, the catalyst separation section 210 may be in fluid communication with the combustor 350 (e.g., via standpipe 426) and the catalyst separation section 310 may be in fluid communication with the upstream reactor section 250 (e.g., via standpipe 424 and transport riser 430).

Generally as is described herein, in embodiments illustrated in FIG. 2, catalyst is cycled between the reactor portion 200 and the catalyst processing portion 300. It should be understood that when “catalysts” are referred to herein, they may refer to solid materials that are catalytically active for a desired reaction. The terms “catalytic activity” and “catalyst activity” refer to the degree to which the catalyst is able to catalyze the reactions conducted in the reactor system 102. The catalyst that exits the reactor portion 200 may be deactivated catalyst. As used herein, “deactivated” may refer to a catalyst which has reduced catalytic activity or is cooler as compared to catalyst entering the reactor portion 200. However, deactivated catalyst may maintain some catalytic activity. Reduced catalytic activity may result from contamination with a substance such as coke. Coke may form on the catalyst within the reactor portion 200. Reactivation (sometimes called “regeneration” herein) may remove the contaminant such as coke, raise the temperature of the catalyst, or both. In embodiments, deactivated catalyst may be reactivated by catalyst reactivation in the catalyst processing portion 300. The deactivated catalyst may be reactivated by, but not limited to, removing coke by combustion, oxidizing the catalyst, other reactivation process, or combinations thereof. In some embodiments, the catalyst may be heated during reactivation by combustion of a supplemental fuel, such as methane, ethane, propane, natural gas, or combinations thereof. The reactivated catalyst from the catalyst processing portion 300 is then passed back to the reactor portion 200.

In non-limiting examples, the reactor system 102 described herein may be utilized to produce light olefins from a hydrocarbon-containing feed. According to one or more embodiments, the reaction may be a dehydrogenation reaction. According to such embodiments, the hydrocarbon-containing feed may comprise one or more of ethane, propane, n-butane, and i-butane. In one or more embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99wt. % of the sum of ethane, propane, n-butane, and i-butane.

In one or more embodiments, the process catalyst may comprise, consist essentially of, or consist of one or more of gallium, indium, or thallium, one or more of platinum, palladium, rhodium, iridium, ruthenium, or osmium, and a support. As described herein, “consisting essentially of” refers to materials with less than 1 wt. % of the non-recited materials (i.e., consisting essentially of A and B means A and B combined are at least 99 wt. % of the composition). As is described herein, the catalyst may be solid particles suitable for fluidization.

In one or more embodiments, the process catalyst may comprise one or more of gallium, indium, or thallium in an amount of from 0.1 wt. % to 10 wt. % based on the total mass of the process catalyst. Such materials may catalyze the dehydrogenation of alkanes to alkenes, particularly when used in combination with one or more of platinum, palladium, rhodium, iridium, ruthenium, or osmium. For example, the process catalyst may comprise one or more of gallium, indium, or thallium in an amount from 0.1 wt. % to 0.25 wt. %, from 0.25 wt. % to 0.5 wt. %, from 0.5 wt. % to 0.75 wt. %, from 0.75 wt. % to 1 wt. %, from 1 wt. % to 2 wt. %, from 2 wt. % to 3 wt. %, from 3 wt. % to 4 wt. %, from 4 wt. % to 5 wt. %, from 5 wt. % to 6 wt. %, from 6 wt. % to 7 wt. %, from 7 wt. % to 8 wt. %, from 8 wt. % to 9 wt. %, from 9 wt. % to 10 wt. %, or any combination of these ranges. In some embodiments, the catalyst may comprise one or more of gallium, indium, or thallium in an amount from 0.1 wt. % to 9 wt. %, from 0.1 wt. % to 8 wt. %, from 0.1 wt. % to 7 wt. %, from 0.1 wt. % to 6 wt. %, or from 0.1 wt. % to 5 wt. %. In some embodiments, the process catalyst comprises only gallium but not indium or thallium, only indium but not gallium or thallium, or only thallium but not gallium or indium. It should be understood that the compositional ranges describing the amount of gallium, indium, and thallium represent ranges for any one of these materials, or for the combination of these materials. Without being bound by theory, it is believed that compositions having one or more of gallium, indium, or thallium in an amount less than 0.1 wt. % negatively impacts the process catalyst's ability to catalyze the alkane dehydrogenation process by lowering both the percentage of total alkane dehydrogenated and the percentage of dehydrogenated alkane that is the intended product. However, it is believed that compositions having one or more of gallium, indium, or thallium in an amount exceeding 10 wt. % may negatively impact the catalyst's ability to catalyze the alkane dehydrogenation process, negatively impact the catalyst's selectivity towards the intended product, or both.

In one or more embodiments, the process catalyst may comprise one or more of platinum, palladium, rhodium, iridium, ruthenium, or osmium in an amount from 5 ppmw to 1000 ppmw based on the total mass of the process catalyst. Such materials may catalyze the dehydrogenation of alkanes to alkenes, particularly when used in combination with one or more of gallium, indium, or thallium. For example, the process catalyst may comprise one or more of platinum, palladium, rhodium, iridium, ruthenium, or osmium in an amount from 5 ppmw to 50 ppmw, from 50 ppmw to 100 ppmw, from 100 ppmw to 200 ppmw, from 200 ppmw to 300 ppmw, from 300 ppmw to 400 ppmw, from 400 ppmw to 500 ppmw, from 500 ppmw to 600 ppmw, from 600 ppmw to 700 ppmw, from 700 ppmw to 800 ppmw, from 800 ppmw to 900 ppmw, from 900 ppmw to 1000 ppmw, or any combination of these ranges. In some embodiments, the process catalyst may comprise one or more of platinum, palladium, rhodium, iridium, ruthenium, or osmium in an amount from 5 ppmw to 900 ppmw, from 5 ppmw to 800 ppmw, from 5 ppmw to 700 ppmw, from 5 ppmw to 600 ppmw, from 5 ppmw to 500 ppmw, or from 10 ppmw to 400 ppmw. In some embodiments, the process catalyst comprises only platinum but not palladium, rhodium, iridium, ruthenium, or osmium, only palladium but not platinum, rhodium, iridium, ruthenium, or osmium, only rhodium, but not platinum palladium, iridium, ruthenium, or osmium, only iridium, but not platinum palladium, rhodium, ruthenium, or osmium, only ruthenium but not platinum, palladium, rhodium, iridium, or osmium, or only osmium but not platinum, palladium, rhodium, iridium, or ruthenium. It should be understood that the compositional ranges describing the amount of platinum, palladium, rhodium, iridium, ruthenium, and osmium represent ranges for any one of these materials, or for the combination of these materials. Without being bound by theory, it is believed that compositions having one or more of platinum, palladium, rhodium, iridium, ruthenium, or osmium in an amount less than 5 ppmw negatively impacts the process catalyst's ability to catalyze the alkane dehydrogenation process by lowering both the percentage of total alkane dehydrogenated and the percentage of dehydrogenated alkane that is the intended product. However, it is believed that compositions having one or more of platinum, palladium, rhodium, iridium, ruthenium, or osmium in an amount exceeding 1000 ppmw may negatively impact the catalyst's ability to catalyze the alkane dehydrogenation process, negatively impact the catalyst's selectivity towards the intended product, or both.

As is described herein, in one or more embodiments, the catalyst may comprise a support. The support may comprise one or more of alumina, silica, or combinations thereof. For example, in some embodiments the support may comprise one or more of alumina, silica-containing alumina, zirconia-containing alumina, titania-containing alumina, and lanthanum-containing alumina. The support may be present in an amount of at least 85 wt. % relative to the total weight of the catalyst, such as at least 85 wt. %, at least 90 wt. %, or at least 95 wt. %. In some embodiments, the support comprises less than or equal to 99.5 wt. % of the catalyst. Generally, the wt. % of the support may fill the remainder of the total catalyst not specified by other materials.

In one or more embodiments, the process catalyst may optionally comprise one or more alkali metals, one or more alkaline earth metals, or both in an amount from 0.01 wt. % to 2.5 wt. % based on the total mass of the catalyst. For example, the process catalyst may comprise one or more alkali metals, one or more alkaline earth metals, or both in an amount from 0.01 wt. % to 0.05 wt. %, form 0.05 wt. % to 0.1 wt. %, from 0.1 wt. % to 0.2 wt. %, from 0.2 wt. % to 0.3 wt. %, from 0.3 wt. % to 0.4 wt. %, from 0.4 wt. % to 0.5 wt. %, from 0.5 wt. % to 0.6 wt. %, from 0.6 wt. % to 0.7 wt. %, from 0.7 wt. % to 0.8 wt. %, from 0.8 wt. % to 0.9 wt. %, from 0.9 wt. % to 1 wt. %, from 1 wt. % to 1.25 wt. %, from 1.25 wt. % to 1.5 wt. %, from 1.5 wt. % to 1.75 wt. %, from 1.75 wt. % to 2 wt. %, from 2 wt. % to 2.25 wt. %, from 2.25 wt. % to 2.5 wt. %, or any combination of these ranges. In some embodiments, the process catalyst may comprise one or more alkali metals, one or more alkaline earth metals, or both from 0.01 wt. % to 1 wt. %, from 0.02 wt. % to 0.75 wt. %, from 0.03 wt. % to 0.5 wt. %, from 0.04 wt. % to 0.4 wt. %, or from 0.05 wt. % to 0.3 wt. %. In some embodiments, the one or more alkali metals or one or more alkaline earth metals may be potassium. Without being bound by theory, it is believed that compositions having alkali metals or alkaline earth metals in an amount less than 0.01 wt. % may cause the production of undesired products during the dehydrogenation reaction. However, it is believed that compositions having alkali metals or alkaline earth metals in an amount exceeding 2.5 wt. % may reduce the catalyst's dehydrogenation activity.

In one or more embodiments, the process catalyst may comprise one or more of iron or manganese in a combined amount from 100 ppmw to 5000 ppmw based on the total mass of the catalyst. For example, the process catalyst may contain one or more of iron or manganese in a combined amount from 100 ppmw to 500 ppmw, from 500 ppmw to 1000 ppmw, from 1000 ppmw to 2000 ppmw, from 2000 ppmw to 3000 ppmw, from 3000 ppmw to 4000 ppmw, from 4000 ppmw to 5000 ppmw, or any combination of these ranges. In some embodiments, the process catalyst my comprise iron but not manganese or manganese but not iron. Without being bound by theory it is believed that compositions having iron or manganese content in an amount exceeding 5000 ppmw may negatively impact the alkane dehydrogenation performance of the catalyst. However, it is believed that compositions having iron or manganese content in an amount less than 100 ppmw may not sufficiently improve the catalysts alkane dehydrogenation performance. In some embodiments, the process catalyst may be void of one or more of iron or manganese.

In one or more embodiments, the process catalyst may include solid particulates that are capable of fluidization. In some embodiments, the process catalyst may exhibit properties known in the industry as “Geldart A” or “Geldart B” properties. Catalyst type may be classified as “Group A” or “Group B” according to D. Geldart, Gas Fluidization Technology, John Wiley & Sons (New York, 1986), 34-37; and D. Geldart, “Types of Gas Fluidization,” Powder Technol. 7 (1973) 285-292, the disclosures of which are incorporated herein by reference in their entireties.

Geldart Group A is understood by those skilled in the art as representing an aeratable powder, having a bubble-free range of fluidization; a high bed expansion; a slow and linear deaeration rate; bubble properties that may include a predominance of splitting/recoalescing bubbles, with a maximum bubble size and large wake; high levels of solids mixing and gas backmixing, assuming equal U-Umf (U is the velocity of the carrier gas, and Umf is the minimum fluidization velocity, typically though not necessarily measured in meters per second, m/s, i.e., there is excess gas velocity); axisymmetric slug properties; and no spouting, except in very shallow beds. The properties listed tend to improve as the mean particle size decreases, assuming equal dp; or as the <45 micrometers (μm) proportion is increased; or as pressure, temperature, viscosity, and density of the gas increase. In general, the particles may exhibit a small mean particle size and/or low particle density (<1.4 grams per cubic centimeter, g/cm³), fluidize easily, with smooth fluidization at low gas velocities, and may exhibit controlled bubbling with small bubbles at higher gas velocities.

Geldart Group B is understood by those skilled in the art as representing a “sand-like” powder that starts bubbling at Umf; that exhibits moderate bed expansion; a fast deaeration; no limits on bubble size; moderate levels of solids mixing and gas backmixing, assuming equal U-Umf; both axisymmetric and asymmetric slugs; and spouting in only shallow beds. These properties tend to improve as mean particle size decreases, but particle size distribution and, with some uncertainty, pressure, temperature, viscosity, or density of gas seem to do little to improve them. In general, most of the particles having a particle size (dp) of 40 μm<dp<500 μm when the density (ρp) is 1.4 <ρp <4 g/cm³.

Still referring again to FIG. 2 the hydrocarbon-containing feed may enter feed inlet 434 into the reactor 202, and the olefin-containing effluent may exit the reactor system 102 via pipe 420. According to one or more embodiments, the reactor system 102 may be operated by feeding a hydrocarbon-containing feed (e.g., in a feed stream) and a fluidized catalyst into the upstream reactor section 250. The hydrocarbon-containing feed contacts the catalyst in the upstream reactor section 250, and each flow upwardly into and through the downstream reactor section 230 to produce an olefin-containing effluent.

Now referring to FIG. 2 in detail, the reactor portion 200 may comprise an upstream reactor section 250, a transition section 258, and a downstream reactor section 230, such as a riser. The transition section 258 may connect the upstream reactor section 250 with the downstream reactor section 230. As depicted in FIG. 1, the upstream reactor section 250 may be positioned below the downstream reactor section 230. Such a configuration may be referred to as an upflow configuration in the reactor 202. The upstream reactor section 250 may include a vessel, drum, barrel, vat, or other container suitable for a given chemical reaction. As depicted in FIG. 2, the upstream reactor section 250 may be connected to the downstream reactor section 230 via the transition section 258. The upstream reactor section 250 may generally comprise a greater cross-sectional area than the downstream reactor section 230. The transition section 258 may be tapered from the size of the cross-section of the upstream reactor section 250 to the size of the cross-section of the downstream reactor section 230 such that the transition section 258 projects inwardly from the upstream reactor section 250 to the downstream reactor section 230. For example, the transition section 258 may be a frustum.

The upstream reactor section 250 may be connected to a transport riser 430, which, in operation may provide reactivated catalyst in a feed stream to the reactor portion 200. The reactivated catalyst and/or reactant chemicals may be mixed with a distributor 260 housed in the upstream reactor section 250. The catalyst entering the upstream reactor section 250 via transport riser 430 may be passed through standpipe 424 to a transport riser 430, thus arriving from the catalyst processing portion 300. In some embodiments, catalyst may come directly from the catalyst separation section 210 via standpipe 422 and into a transport riser 430, where it enters the upstream reactor section 250, where in such embodiments some of the catalyst is not passed through the catalyst processing portion 300. The catalyst can also be fed via standpipe 422 directly to the upstream reactor section 250 (not depicted in FIG. 2). This catalyst may be somewhat deactivated, but may still, in some embodiments, be suitable for reaction in the upstream reactor section 250, particularly when used in combination with reactivated catalyst.

In one or more embodiments, the catalyst may have a residence time within the reactor portion 200 of less than or equal to 3 minutes. As the term is used herein, “residence time” refers to the average amount of time the catalyst or other specified material spends within the reactor portion 200. As it is an average, the amount of time the catalyst may spend within the reactor portion 200 during any given cycle may have a distribution and not be equal to the average, but over time will average out to be equal to about the residence time. In some embodiments, the catalyst may have a residence time within the reactor portion 200 of less than or equal to 2.5 min., less than or equal to 2 min., less than or equal to 1.5 min., less than or equal to 1 min., less than or equal to 0.5 min., or less than or equal to 0.1 min. Without being bound by theory, it is believed that catalyst residence time greater than 3 minutes may increase equipment costs without a matching increase in catalyst dehydrogenation performance. However, it is believed that catalyst residence time less than 0.1 minutes may not allow the catalyst to sufficiently catalyze the dehydrogenation reaction.

Still referring to FIG. 2, in one or more embodiments, based on the shape, size, and other processing conditions (such as temperature and pressure) in the upstream reactor section 250 and the downstream reactor section 230, the upstream reactor section 250 may operate as a fluidized bed, such as in a fast fluidized, turbulent, or bubbling bed upflow reactor, while the downstream reactor section 230 may operate in more of a plug flow manner, such as in a riser reactor. For example, the reactor 202 of FIG. 2 may comprise an upstream reactor section 250 operating as a fast fluidized, turbulent, or bubbling bed reactor and a downstream reactor section 230 operating as a dilute phase riser reactor, with the result that the average catalyst and gas flow moves concurrently upward. As the term is used herein, “average flow” refers to the net flow, i.e., the total upward flow minus the retrograde or reverse flow, as is typical of the behavior of fluidized particles in general. As described herein, a “fast fluidized” reactor may refer to a reactor utilizing a fluidization regime wherein the superficial velocity of the gas phase is greater than the choking velocity and may be semi-dense in operation. As described herein, a “turbulent” reactor may refer to a fluidization regime where the superficial velocity of less than the choking velocity and is more dense than the fast fluidized regime. As described herein, a “bubbling bed” reactor may refer to a fluidization regime wherein well defined bubbles in a highly dense bed are present in two distinct phases. The “choking velocity” refers to the minimum velocity required to maintain solids in the dilute-phase mode in a vertical conveying line. As described herein, a “dilute phase riser” may refer to a riser reactor operating at above choking velocity.

According to embodiments, the olefin-containing effluent and the catalyst may be passed out of the downstream reactor section 230 to a separation device 220 in the catalyst separation section 210, where the catalyst is at least partially separated from the olefin-containing effluent, which is transported out of the catalyst separation section 210. According to one or more embodiments, following separation from vapors in the separation device 220, the catalyst may generally move through the stripper 224 to the catalyst outlet port 222 where the catalyst is transferred out of the reactor portion 200 via standpipe 426 and into the catalyst processing portion 300.

According to one or more embodiments, the separation device 220 may be a cyclonic separation system, which may include two or more stages of cyclonic separation. In embodiments where the separation device 220 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the catalyst from the product gas. It should be understood that any primary cyclonic separation device may be used in embodiments of the present disclosure.

Still referring to FIG. 2, the separated catalyst is passed from the catalyst separation section 210 to the combustor 350. In the combustor 350, the catalyst may be processed by, for example, combustion of coke with oxygen. For example, and without limitation, the catalyst may be de-coked and/or supplemental fuel may be combusted to heat the catalyst. The catalyst is then passed out of the combustor 350 and through the riser 330 to a riser termination separator 378, where the gas and solid components from the riser 330 are at least partially separated. The vapor and remaining solids are transported to a secondary separation device 320 in the catalyst separation section 310 where the remaining catalyst is separated from the gases from the catalyst processing (e.g., gases emitted by combustion of spent catalyst or supplemental fuel, referred to herein as flue gas). The flue gas may pass out of the catalyst processing portion 300 via outlet pipe 432. The separated catalyst is then passed through the oxygen treatment zone 370 within the catalyst separation section 310 to the upstream reactor section 250 via standpipe 424 and transport riser 430, where it is further utilized in a catalytic reaction. Thus, the catalyst, in operation, may cycle between the reactor portion 200 and the catalyst processing portion 300. In general, the processed chemical streams, including the hydrocarbon-containing feed and olefin-containing effluent may be gaseous, and the catalyst may be fluidized particulate solid.

Referring now to the catalyst processing portion 300, as depicted in FIG. 2, the combustor 350 of the catalyst processing portion 300 may include one or more lower reactor portion inlet ports 352 and may be in fluid communication with the riser 330. Oxygen-containing gas, such as air, may be passed through pipe 428 into the combustor 350. The combustor 350 may be in fluid communication with the catalyst separation section 210 via standpipe 426, which may supply spent catalyst from the reactor portion 200 to the catalyst processing portion 300 for regeneration. The combustor 350 and riser 330, collectively referred to as the catalyst combustion reactor 302, may operate with similar or identical fluidization regimes as to what was disclosed with respect to the upstream reactor section 250 and downstream reactor section 230 of the reactor portion 200. That is, the combustor 350 may operate as a fluidized bed, such as in a fast fluidized, turbulent, or bubbling bed upflow reactor, while the riser 330 may operate in more of a plug flow manner, such as in a riser reactor. Geometries as described with respect to the upstream reactor section 250 and downstream reactor section 230 may equally apply to the combustor 350 and riser 330. Additionally, the combustor 350 may also include a fuel inlet 354, which may supply a fuel, such as a hydrocarbon stream, to the combustor 350.

As described herein, the catalyst may be heated in the catalyst processing portion 300 by combustion of supplemental fuels. Supplemental fuels may combust with oxygen to heat the catalyst, and supplemental fuels such as a supplemental fuel, such as hydrogen, methane, ethane, propane, natural gas, or combinations thereof. Without being bound by any theory, when methane is utilized in the supplemental fuel, catalysts as described herein that have been modified may better catalyze the combustion of methane to heat the catalyst. Catalysts which have not been modified, when methane is utilized in the supplemental fuel, may be deficient by not promoting heating of the catalyst to a temperature needed for dehydrogenation.

As described in one or more embodiments, following separation of flue gas from catalyst in the riser termination separator 378 and secondary separation device 320, treatment of the processed catalyst with an oxygen-containing gas is conducted in the oxygen treatment zone 370. In some embodiments, the oxygen treatment zone 370 includes a fluid solids contacting device. The fluid solids contacting device may include baffles or grid structures to facilitate contact of the processed catalyst with the oxygen-containing gas. Examples of fluid solid contacting devices are described in further detail in U.S. Pat. Nos. 9,827,543 and 9,815,040. The fluidization regime within the oxygen treatment zone may be bubbling bed type fluidization. The oxygen treatment zone 370 may include an oxygen-containing gas inlet 372, which may supply an oxygen-containing gas to the oxygen treatment zone 370 for oxygen treatment of the catalyst.

As is disclosed herein, in one or more embodiments the catalyst may be exposed to an oxygen-containing gas in oxygen treatment zone 370. For example, the catalyst may be exposed to an oxygen-containing gas for from 2 min. to 20 min., such as from 2 min. to 4 min., from 4 min. to 6 min., from 6 min. to 8 min., from 8 min. to 10 min., from 10 min. to 12 min., from 12 min. to 14 min., from 14 min. to 16 min., from 16 min. to 18 min., from 18 min. to 20 min., or any combination of these ranges. In some embodiments the catalyst may be exposed to an oxygen containing gas from 4 min. to 18 min., from 6 min. to 17 min., from 8 min. to 16 min., or from 10 min. to 15 min. Without being bound by theory, it is believed that exposure of the catalyst to an oxygen-containing gas for more than 20 minutes may increase equipment costs without a matching increase in catalyst regeneration efficiency. However, it is believed that oxygen-containing gas exposure for less than 2 minutes may lead to less efficient regeneration of the catalyst which may reduce the catalyst's dehydrogenation activity.

In one or more embodiments, the light olefins may be present in a “product stream” sometimes called an “olefin-containing effluent” and include light olefins. Such a stream exits the reactor system 102 of FIG. 2 and may be subsequently processed. As used in the present disclosure, the term “light olefins” refers to one or more of ethylene, propylene, and butene. The term butene includes any isomers of butene, such as α-butylene, cis-β-butylene, trans-β-butylene, and isobutylene. In some embodiments, the olefin-containing effluent includes at least 25 wt. % light olefins based on the total weight of the olefin-containing effluent. For example, the olefin-containing effluent may include at least 35 wt. % light olefins, at least 45 wt. % light olefins, at least 55 wt. % light olefins, at least 65 wt. % light olefins, or at least 75 wt. % light olefins based on the total weight of the olefin-containing effluent. The olefin-containing effluent may further comprise unreacted components of the hydrocarbon-containing effluent, as well as other reaction products that are not considered light olefins. The light olefins may be separated from unreacted components in subsequent separation steps.

Now referring again to FIG. 1, step 120 generally includes withdrawing the process catalyst from the dehydrogenation process. Over time the process catalyst may degrade while in use within the dehydrogenation process. This degradation may include the loss of catalytically active components, such as palladium or platinum from the process catalyst or the deactivation of the catalytic components of the process catalyst. For conventional catalysts this loss of catalytic materials will eventually require the process catalyst to be replaced with fresh catalyst. The replacement of the process catalyst with fresh catalyst may be wasteful as many of the components of the process catalyst, such as the catalytic materials and the support remain functional. As will be described herein, modifying the process catalyst may allow for the reuse of functional catalyst materials which may reduce waste production.

In one or more embodiments, the withdrawing of the process catalyst may be done in bulk, such that the dehydrogenation process is stopped and a significant portion of the process catalyst present in the dehydrogenation process is removed. For example, the amount of catalyst withdrawn may be at least 5% of the total catalyst present, at least 10%, at least 20%, at least 30%, at least 40%, at least 50%, at least 60%, at least 70%, at least 80%, at least 90%, or even 100% of the total catalyst present may be withdrawn.

In one or more embodiments, the process catalyst is continuously withdrawn. As used in the present disclosure, the term “continuously withdrawn” means the withdrawal of the process catalyst occurs such that some of the process catalyst is being withdrawn from the dehydrogenation process consistently so as to not require the dehydrogenation process to stop in order to withdraw the process catalyst. For example, continuous withdrawal may be accomplished by installation of catalyst withdrawal systems, such as the Johnson Matthey INTERCAT™ continuous Catalyst Withdrawal System. In some embodiments, the amount of process catalyst continuously withdrawn and the amount of catalyst added back to the dehydrogenation process is determined by measuring the catalyst loss from unit through mechanical attrition and the rate of performance of the process catalyst as it ages, so the process catalyst may be continuously withdrawn to maintain performance. In some embodiments, when the process catalyst is continuously withdrawn at least 0.05% of the process catalyst may be withdrawn from the dehydrogenation process. For example, at least 0.1% of the process catalyst may be withdrawn from the dehydrogenation process, at least 0.25% of the process catalyst, at least 0.5% of the process catalyst, at least 1.0% of the process catalyst, at least 1.5% of the process catalyst, at least 2.0% of the process catalyst, at least 2.5% of the process catalyst, at least 3.0% of the process catalyst, at least 3.5% of the process catalyst, at least 4.0% of the process catalyst, at least 4.5% of the process catalyst, or even up to 5.0% of the process catalyst may be continuously withdrawn.

In one or more embodiments, the process catalyst may be withdrawn when there is a decrease in dehydrogenation or combustion activity such that the targeted productivity (e.g. olefin production rate) or combustion activity can no longer be achieved by adding fresh catalyst to compensate for the mechanical loss of catalyst from the unit or by applying high severity operation conditions, such as higher reaction temperature, higher regeneration temperature, or changing the catalyst to oil ratio. For example, the process catalyst may be withdrawn when combustion activity is not sufficient to reach at least 5% lower flammability limit (LFL), at least 10% LFL, at least 20% LFL, or at least 40% LFL, or the process catalyst may be withdrawn in bulk when productivity drops to 95% of the nameplate productivity, 90% of the nameplate productivity, 85% of the nameplate productivity, or 80% of the nameplate productivity. As used in the present disclosure, the term “lower flammability limit” refers to the lower end of the concentration range over which a flammable mixture of gas or vapor in air can be ignited at a given temperature and pressure. The LFL of the combustion gases may be determined by reactive chemistry testing or as described by Michael G. Zabetakis, Flammability Characteristics of Combustible Gases and Vapors, 627 Bureau of Mines 1 (1965), with pressure adjustments according to Coward et al., Limits of Flammability of Gases and Vapors, 503 Bureau of Mines 1 (1952). In some embodiments, when mechanical loss of catalyst is low and as such does not provide enough replacement space to allow addition of catalyst to compensate for the loss in dehydrogenation and combustion activity, catalyst may be intentionally withdrawn from the unit on a routine basis to provide replacement space to allow for the addition of catalyst.

In one or more embodiments, the process catalyst may be withdrawn after being used in the dehydrogenation process for a given amount of time. For example, the process catalyst may be withdrawn after at least 6 months of dehydrogenation process cycles, such as after at least 2 weeks, after at least 1 month, after at least 2 months, after at least 3 months, after at least 4 months, after at least 5 months, after at least 7 months, after at least 8 months, after at least 9 months, after at least 10 months, after at least 11 months, or after at least 1 year.

Now referring again to FIG. 1, step 130 generally includes modifying the process catalyst to form a modified catalyst. As used in the present disclosure the term “modified catalyst” refers to a process catalyst that has been modified through the addition of one or more metals. In one or more embodiments, the process catalyst may be modified by adding one or more of manganese, iron, chromium, vanadium, or aluminum to the process catalyst to form a modified catalyst.

In one or more embodiments, the modified catalyst may comprise manganese in an amount from 100 ppmw to 5000 ppmw based on the total mass of the modified catalyst. For example, the modified catalyst may comprise manganese in an amount from 100 ppmw to 500 ppmw, from 500 ppmw to 1000 ppmw, from 1000 ppmw to 2000 ppmw, from 2000 ppmw to 3000 ppmw, from 3000 ppmw to 4000 ppmw, from 4000 ppmw to 5000 ppmw, or any combination of these ranges. In some embodiments, the modified catalyst may comprise manganese in an amount from 500 ppmw to 4500 ppmw, from 750 ppmw to 4000 ppmw, from 1000 ppmw to 3500 ppmw, or from 1500 ppmw to 3000 ppmw. Without being bound by theory, it is believed that compositions having manganese in an amount exceeding 5000 ppmw may negatively impact the catalyst's ability to catalyze the alkane dehydrogenation process by lowering both the percentage of total alkane dehydrogenated and the percentage of dehydrogenated alkane that is the intended product. However, it is believed that compositions having manganese in an amount less than 100 ppmw manganese may not sufficiently recover alkane conversion and fuel gas combustion activity when compared to an unmodified process catalyst.

In one or more embodiments, the modified catalyst may comprise iron in an amount from 100 ppmw to 5000 ppmw based on the total mass of the modified catalyst. For example, the modified catalyst may comprise iron in an amount from 100 ppmw to 500 ppmw, from 500 ppmw to 1000 ppmw, from 1000 ppmw to 2000 ppmw, from 2000 ppmw to 3000 ppmw, from 3000 ppmw to 4000 ppmw, from 4000 ppmw to 5000 ppmw, or any combination of these ranges. In some embodiments, the modified catalyst may comprise iron in an amount from 500 ppmw to 4500 ppmw, from 750 ppmw to 4000 ppmw, from 1000 ppmw to 3500 ppmw, or from 1500 ppmw to 3000 ppmw. Without being bound by theory, it is believed that compositions having iron in an amount less than 100 ppmw may not sufficiently recover alkane conversion and fuel gas combustion activity when compared to an unmodified process catalyst. However, it is believed that compositions having iron in an amount exceeding 5000 ppmw may negatively impact the catalyst's ability to catalyze the alkane dehydrogenation process, negatively impact the catalyst's selectivity towards the intended product, or both.

In one or more embodiments, the modified catalyst may comprise chromium in an amount from 100 ppmw to 5000 ppmw based on the total mass of the modified catalyst. For example, the modified catalyst may comprise chromium in an amount from 100 ppmw to 500 ppmw, from 500 ppmw to 1000 ppmw, from 1000 ppmw to 2000 ppmw, from 2000 ppmw to 3000 ppmw, from 3000 ppmw to 4000 ppmw, from 4000 ppmw to 5000 ppmw, or any combination of these ranges. In some embodiments, the modified catalyst may comprise chromium in an amount from 500 ppmw to 4500 ppmw, from 750 ppmw to 4000 ppmw, from 1000 ppmw to 3500 ppmw, or from 1500 ppmw to 3000 ppmw. Without being bound by theory, it is believed that compositions having chromium in an amount less than 100 ppmw may not sufficiently recover alkane conversion and fuel gas combustion activity when compared to an unmodified process catalyst. However, it is believed that compositions having chromium in an amount exceeding 5000 ppmw may negatively impact the catalyst's ability to catalyze the alkane dehydrogenation process, negatively impact the catalyst's selectivity towards the intended product, or both.

In one or more embodiments, the modified catalyst may comprise vanadium in an amount from 1 ppmw to 2000 ppmw based on the total mass of the modified catalyst. For example, the modified catalyst may comprise vanadium in an amount from 1 ppmw to 100 ppmw, from 100 ppmw to 500 ppmw, from 500 ppmw to 1000 ppmw, from 1000 ppmw to 1500 ppmw, from 1500 ppmw to 2000 ppmw, or any combination of these ranges. In some embodiments, the modified catalyst may comprise from vanadium in an amount from 100 ppmw to 1900 ppmw, from 500 ppmw to 1800 ppmw, from 750 ppmw to 1700 ppmw, from 1000 ppmw to 1600 ppmw, from 1100 ppmw to 1500 ppmw, from 1200 ppmw to 1450 ppmw, or from 1300 ppmw to 1400 ppmw. Without being bound by theory, it is believed that compositions having vanadium in an amount less than 1 ppmw may not sufficiently recover alkane conversion and fuel gas combustion activity when compared to an unmodified process catalyst. However, it is believed that compositions having vanadium in an amount exceeding 2000 ppmw may negatively impact the catalyst's ability to catalyze the alkane dehydrogenation process, negatively impact the catalyst's selectivity towards the intended product, or both.

In one or more embodiments, the modified catalyst may comprise aluminum in an amount from 0.5 wt. % to 10 wt. % based on the total mass of the modified catalyst not including aluminum in the form of aluminum oxide that may be present as part of the support. For example, the modified catalyst may comprise aluminum from 0.5 wt. % to 1 wt. %, from 1 wt. % to 2 wt. %, from 2 wt. % to 3 wt. %, from 3 wt. % to 4 wt. %, from 4 wt. % to 5 wt. %, from 5 wt. % to 6 wt. %, from 6 wt. % to 7 wt. %, from 7 wt. % to 8 wt. %, from 8 wt. % to 9 wt. %, from 9 wt. % to 10 wt. %, or any combination of these ranges. In some embodiments, the modified catalyst may comprise aluminum in an amount from 0.75 wt. % to 8 wt. %, from 1 wt. % to 7 wt. %, from 1.5 wt. % to 6 wt. %, or from 2 wt. % to 5 wt. %. Without being bound by theory, it is believed that compositions having aluminum in an amount less than 0.5 wt. % may not sufficiently recover alkane conversion and fuel gas combustion activity when compared to an unmodified process catalyst. However, it is believed that compositions having aluminum in an amount exceeding 10 wt. % may negatively impact the catalyst's ability to catalyze the alkane dehydrogenation process, negatively impact the catalyst's selectivity towards the intended product, or both.

In one or more embodiments the process catalyst may be modified by adding one or more of platinum or gallium to create a modified catalyst. In some embodiments one or more of platinum or gallium may be added to the process catalyst in addition to one or more of manganese, iron, chromium, vanadium, or aluminum. For example, in some embodiments the process catalyst may be modified by adding platinum and iron, platinum and manganese, platinum and chromium, platinum and vanadium, platinum and aluminum, gallium and iron, gallium and manganese, gallium and chromium, gallium and vanadium, gallium and aluminum, platinum, gallium and iron, platinum, gallium and manganese, platinum, gallium, and chromium, platinum, gallium and vanadium, or platinum, gallium and aluminum.

In one or more embodiments, the modified catalyst may comprise platinum an amount from 5 ppmw to 1000 ppmw based on the total mass of the modified catalyst. For example, the modified catalyst may comprise platinum in an amount from 5 ppmw to 50 ppmw, from 50 ppmw to 100 ppmw, from 100 ppmw to 200 ppmw, from 200 ppmw to 300 ppmw, from 300 ppmw to 400 ppmw, from 400 ppmw to 500 ppmw, from 500 ppmw to 600 ppmw, from 600 ppmw to 700 ppmw, from 700 ppmw to 800 ppmw, from 800 ppmw to 900 ppmw, from 900 ppmw to 1000 ppmw, or any combination of these ranges. In some embodiments, the modified catalyst may comprise platinum in an amount from 5 ppmw to 900 ppmw, from 5 ppmw to 800 ppmw, from 5 ppmw to 600 ppmw, from 5 ppmw to 500 ppmw, or from 10 ppmw to 400 ppmw. Without being bound by theory, it is believed that compositions having platinum in an amount less than 5 ppmw negatively impacts the catalyst's ability to catalyze the alkane dehydrogenation process by lowering both the percentage of total alkane dehydrogenated and the percentage of dehydrogenated alkane that is the intended product. However, it is believed that compositions having platinum in an amount exceeding 1000 ppmw may negatively impact the catalyst's ability to catalyze the alkane dehydrogenation process, negatively impact the catalyst's selectivity towards the intended product, or both.

In one or more embodiments, the modified catalyst may comprise gallium in an amount of from 0.1 wt. % to 10 wt. % based on the total mass of the modified catalyst. For example, the modified catalyst may comprise gallium in an amount from 0.1 wt. % to 0.25 wt. %, from 0.25 wt. % to 0.5 wt. %, from 0.5 wt. % to 0.75 wt. %, from 0.75 wt. % to 1 wt. %, from 1 wt. % to 2 wt. %, from 2 wt. % to 3 wt. %, from 3 wt. % to 4 wt. %, from 4 wt. % to 5 wt. %, from 5 wt. % to 6 wt. %, from 6 wt. % to 7 wt. %, from 7 wt. % to 8 wt. %, from 8 wt. % to 9 wt. %, from 9 wt. % to 10 wt. %, or any combination of these ranges. In some embodiments, the modified catalyst may comprise one or more of gallium, indium, or thallium in an amount from 0.1 wt. % to 9 wt. %, from 0.1 wt. % to 8 wt. %, from 0.1 wt. % to 7 wt. %, from 0.1 wt. % to 6 wt. %, or from 0.1 wt. % to 5 wt. %. Without being bound by theory, it is believed that compositions having gallium in an amount less than 0.1 wt. % negatively impacts the catalyst's ability to catalyze the alkane dehydrogenation process by lowering both the percentage of total alkane dehydrogenated and the percentage of dehydrogenated alkane that is the intended product. However, it is believed that compositions having gallium in an amount exceeding 10 wt. % may negatively impact the catalyst's ability to catalyze the alkane dehydrogenation process, negatively impact the catalyst's selectivity towards the intended product, or both.

In one or more embodiments, the process catalyst may be modified to form a modified catalyst via incipient wetness impregnation also known as dry impregnation or capillary impregnation. For example, such a process is described in Marceau et al., Impregnation and Drying, Synthesis of Solid Catalysts 59 (2008), which is incorporated herein by reference in its entirety. For example, the process catalyst may be impregnated using metal precursors, then dried at temperatures less than 200° C., and then calcined at temperatures less than 800° C. to produce the catalyst. For example, in some embodiments, the process catalyst may be modified to form a modified catalyst by impregnating the process catalyst with one or more of gallium, platinum, iron, manganese, chromium, vanadium or aluminum, drying the support, and calcining the support. Additionally, other suitable methods for making the catalysts as described herein are contemplated, as would be known by those skilled in the art.

Referring again to FIG. 1, step 140 generally includes adding the modified catalyst back to the dehydrogenation process. The modified catalyst may then cycle through the steps of the method of making light olefins 100 by being utilized in step 110 to become a process catalyst, withdrawn in step 120, modified in step 130 to become a modified catalyst, and added back to the dehydrogenation process in step 140.

### EXAMPLES

The various embodiments of the present disclosure will be further clarified by the following examples. The examples are illustrative in nature and should not be understood to limit the subject matter of the present disclosure.

### Example 1—Additions of Platinum and Gallium

In Example 1, seven different samples of catalytically active particles were prepared and tested. Performance testing for all samples was conducted in a lab-scale fixed bed reactor using simulated reaction-combustion-reactivation cycles in a fixed-bed rig. The samples were tested at ambient pressure under conditions of 0.5 grams (g) of the samples was mixed with 1.0 g of inert silicon carbide and loaded into a quartz reactor. The standard reaction combustion reactivation cycle was performed in three steps. First, dehydrogenation was performed at 625° C. with a feed composition of 90% propane/10% nitrogen and weight hourly space velocity “WHSV” of propane of 10 hr⁻¹ for 60 seconds. Second, combustion was performed at 730° C. under 2.5 mol % Methane/balance air with a total flow of 50 standard cubic centimeters per minute (sccm) and a WHSV of methane of 0.1 hr⁻¹ for 3 minutes. Finally, the reactivation step was performed at 730° C. under 100% air with a flow rate of 40 sccm for 2 minutes. The dehydrogenation performance data was collected at 30 seconds time on stream and the combustion data was collected at 60 seconds time on stream. Performance data after cycle 25 is reported in Table 1.

Comparative Example A was prepared by first preparing microspheroidal alumina support by spray drying a mixture of hydrated alumina and Ludox® Silica and then heating the resulting spray dried particles at a temperature of at least 1000° C. sufficient to achieve particles with particle size ranging from 5 μm to 300 μm, pore volume of 0.20 ±0.10 ml/g, surface area of 70 ±20 m²/g, and silica content 2.5 ±2.5 wt. %. The catalyst material was then prepared using an incipient wetness impregnation method to load the designated metal or metals to the support using nitrate or amine nitrate metal precursors followed by drying at a temperature less than 200° C., and then calcination at a temperature less than 800° C. The exact compositions of the sample is provided in Table 1.

Comparative Example B was created by operating Comparative Example A in a fluidized reactor-regenerator at between 600° C. to 800° C. and under alkane dehydrogenation, methane combustion, and air regeneration conditions for 9 months. Chemical properties and propane dehydrogenation performance of Comparative Example B is listed in Table 1.

Samples 1-4 were prepared via incipient wetness impregnation by adding designated metal or metals to Comparative Example B using nitrate or amine nitrate metal precursors followed by drying and calcination as Comparative Example A.. The amount of metals added for each of these samples and their dehydrogenation and combustion performance is listed in Table 1.

TABLE 1

| | | | | Propane | | Methane |
|---|---|---|---|---|---|---|
| | | | | conver- | Propylene | conver- |
| | Pt | Ga | K | sion | selectivity | sion |
| Sample | [ppm] | [wt. %] | [wt. %] | [%] | [%] | [%] |
| A | 300 | 1.6 | 0.25 | 39.4 | 98.7 | 85.2 |
| B | 80 | 1.5 | 0.23 | 33.5 | 98.9 | 68.4 |

| | | | | |
|---|---|---|---|---|
| 1 | Sample B + Pt (50 ppm) | 36.5 | 98.8 | 75.6 |
| 2 | Sample B + Pt (100 ppm) | 38.1 | 98.7 | 83.8 |
| 3 | Sample B + Pt (220 ppm) | 35.9 | 98.4 | 85.5 |
| 4 | Sample B + Pt (50 ppm) + | 38.0 | 98.8 | 77.4 |
| | Ga (0.3 wt %) | | | |

Table 1 indicates that, Comparative Example B has reduced propane conversion and methane conversion when compared to Comparative Example A, which has not been aged. Table 1 also indicates that samples with added platinum or platinum and gallium such as, Samples 1-4, have improved propane conversion and methane conversion when compared to Comparative Example B. This indicates that the addition of additional platinum and gallium to a used catalyst, such as Comparative Example B, can recover at least a portion of the used catalyst's lost methane and propane conversion.

### Example 2—Addition of Transition Metals

In Example 2, eight different samples of catalytically active particles were prepared and tested. Samples 5-12 were prepared via incipient wetness impregnation by adding designated metal or metals to Comparative Example B using nitrate or amine nitrate metal precursors followed by drying and calcination as in Comparative Example A. The amount of metals added for each of these samples and their dehydrogenation and combustion performance is listed in Table 2.

TABLE 2

| | | | | Propane | | Methane |
|---|---|---|---|---|---|---|
| | | | | conver- | Propylene | conver- |
| | Pt | Ga | K | sion | selectivity | sion |
| Sample | [ppm] | [wt. %] | [wt. %] | [%] | [%] | [%] |
| A | 300 | 1.6 | 0.25 | 39.4 | 98.7 | 85.2 |
| B | 80 | 1.5 | 0.23 | 33.5 | 98.9 | 68.4 |

| | | | | |
|---|---|---|---|---|
| 5 | Sample B + Fe (1520 ppm) | 43.6 | 98.8 | 88.6 |
| 6 | Sample B + Mn (1500 ppm) | 38.7 | 98.8 | 90.6 |
| 7 | Sample B + V (1390 ppm) | 36.7 | 98.5 | 70.2 |
| 8 | Sample B + Ni (1600 ppm) | 30.8 | 97.1 | 74.5 |
| 9 | Sample B + Co (1610 ppm) | 33.8 | 98.9 | 72.9 |
| 10 | Sample B + Cu (1740 ppm) | 24.0 | 96.9 | 98.0 |
| 11 | Sample B + Mo (2620 ppm) | 21.5 | 95.7 | 63.7 |
| 12 | Sample B + Cr (3000 ppm) | 34.4 | 98.4 | 96.5 |

As Table 2 indicates, the addition of a transition metal not present in the original catalyst may improve methane conversion and propane conversion of the catalyst when compared to an aged catalyst such as Comparative Example B. For example, Samples 5-7, 9, and 12 have increased propane conversion and methane conversion when compared to Comparative Example B. Some samples (i.e., Samples 8, and 10) have only increased conversion of methane when compared to Comparative Example B. Sample 11 has no increase in either propane conversion or methane conversion when compared to Comparative Example B. That is, Table 3 indicates that the addition of only some transition metals improves the methane conversion, propane conversion or both of a spent catalyst, such as Comparative Example B.

### Example 3—Addition of Iron or Manganese

In Example 3, seven different samples of catalytically active particles were prepared and tested. Samples 13-19 were prepared via incipient wetness impregnation by adding designated metal or metals to Comparative Example B using nitrate or amine nitrate metal precursors followed by drying and calcination as Comparative Example A.. The amount of metals added for each of these samples and their dehydrogenation and combustion performance is listed in Table 3.

TABLE 3

| | | | | Propane | | Methane |
|---|---|---|---|---|---|---|
| | | | | conver- | Propylene | conver- |
| | Pt | Ga | K | sion | selectivity | sion |
| Sample | [ppm] | [wt. %] | [wt. %] | [%] | [%] | [%] |
| A | 300 | 1.6 | 0.25 | 39.4 | 98.7 | 85.2 |
| B | 80 | 1.5 | 0.23 | 33.5 | 98.9 | 68.4 |

| | | | | |
|---|---|---|---|---|
| 13 | Sample B + Mn (500 ppm) | 35.4 | 98.1 | 85.1 |
| 14 | Sample B + Mn (1500 ppm) | 38.7 | 98.7 | 90.6 |
| 15 | Sample B + Mn (3000 ppm) | 39.2 | 98.9 | 98.2 |
| 16 | Sample B + Fe (500 ppm) | 39.2 | 98.9 | 79.9 |
| 17 | Sample B + Fe (1520 ppm) | 43.6 | 98.8 | 88.6 |
| 18 | Sample B + Fe (3000 ppm) | 42.2 | 98.5 | 97.4 |
| 19 | Sample B + Fe (10000 ppm) | 13.9 | 95.8 | 99.9 |

As Table 3 indicates, samples modified with one of iron or manganese, such as Samples 13-18, have improved propane conversion and methane conversion when compared to Comparative Example B, which is void of iron or manganese. Table 3 also indicates that samples with high iron loading, such as Sample 19, have improved methane conversion, but not improved propane conversion when compared to Comparative Example B.

### Example 4—Addition of Platinum and another Metal

In Example 4, seventeen different samples of catalytically active particles were prepared and tested. Samples 20-36 were prepared were prepared via incipient wetness impregnation by adding designated metal or metals to Comparative Example B using nitrate or amine nitrate metal precursors followed by drying and calcination as Comparative Example A.. The amount of metals added for each of these samples and their dehydrogenation and combustion performance is listed in Table 4.

TABLE 4

| | | | | Propane | Propylene | Methane |
|---|---|---|---|---|---|---|
| | Pt | Ga | K | conversion | selectivity | conversion |
| Sample | [ppm] | [wt. %] | [wt. %] | [%] | [%] | [%] |
| A | 300 | 1.6 | 0.25 | 39.4 | 98.7 | 85.2 |
| B | 80 | 1.5 | 0.23 | 33.5 | 98.9 | 68.4 |

| | | | | |
|---|---|---|---|---|
| 20 | Sample B + Pt (220 ppm) + Mn (3000 ppm) | 39.2 | 98.8 | 99.6 |
| 21 | Sample B + Pt (220 ppm) + Mn (1500 ppm) | 40.8 | 99.0 | 96.6 |
| 22 | Sample B + Pt (220 ppm) + Mn (500 ppm) | 37.9 | 98.4 | 94.1 |
| 23 | Sample B + Pt (100 ppm) + Mn (3000 ppm) | 39.5 | 98.6 | 99.5 |
| 24 | Sample B + Pt (100 ppm) + Mn (1500 ppm) | 38.4 | 98.3 | 97.4 |
| 25 | Sample B + Pt (50 ppm) + Mn (3000 ppm) | 39.6 | 98.9 | 98.3 |
| 26 | Sample B + Pt (50 ppm) + Mn (1500 ppm) | 40.4 | 98.6 | 93.1 |
| 27 | Sample B + Pt (220 ppm) + Fe (3000 ppm) | 38.5 | 98.8 | 98.0 |
| 28 | Sample B + Pt (220 ppm) + Fe (1500 ppm) | 41.5 | 98.9 | 93.1 |
| 29 | Sample B + Pt (220 ppm) + Fe (5000 ppm) | 24.8 | 98.1 | 99.2 |
| 30 | Sample B + Pt (220 ppm) + Fe (10000 ppm) | 10.1 | 93.1 | 99.9 |
| 31 | Sample B + Pt (220 ppm) + Fe (30000 ppm) | 5.8 | 76.0 | 100 |
| 32 | Sample B + Pt (220 ppm) + Cr (3000 ppm) | 37.9 | 98.9 | 98.4 |
| 33 | Sample B + Pt (220 ppm) + Fe (1500 ppm) + | 34.8 | 98.8 | 97.8 |
| | Mn (1500 ppm) | | | |
| 34 | Sample B + Fe (3000 ppm) + Mn (1500 ppm) | 37.4 | 98.8 | 95.0 |
| 35 | Sample B + Pt (220 ppm) + Al (2 wt. %) | 38.1 | 98.7 | 93.3 |
| 36 | Sample B + Pt (220 ppm) + Al (5 wt. %) | 35.4 | 98.3 | 87.8 |

As Table 4 indicates, all samples that were modified by adding both platinum and manganese, such as Samples 20-26 have both improved propane conversion and methane conversion when compared to Comparative Example B, which is not modified, across a wide range of possible modifications, such as from 50 ppmw to 220 ppmw of added platinum and from 500 ppmw to 3000 ppmw of added manganese.

Table 4 also indicates that all samples that were modified by adding both platinum and iron, such as Samples 27-31, have improved methane conversion when compared to Comparative Example B, which is not modified. Some samples that were modified by adding both platinum and iron, such as Samples 27 and 28, have improved methane conversion and propane conversion when compared to Comparative example B. Further, Sample 33 which was modified by adding platinum, manganese, and iron and Sample 34 which was modified by adding manganese and iron both have improved propane conversion and methane conversion when compared to Comparative Example B.

Table 4 further indicates that Sample 32 which was modified by adding both platinum and chromium has improved propane conversion and methane conversion when compared to Comparative Example B. Finally, Table 4 indicates that samples that were modified by adding both platinum and aluminum, such as Samples 35 and 36 have improved propane conversion and methane conversion when compared to Comparative Example B.

In a first aspect of the present disclosure a method may comprise operating a dehydrogenation process whereby a hydrocarbon-containing feed is converted to light olefins, wherein the dehydrogenation process utilizes a fluidized process catalyst that circulates between a reactor and a combustor. Withdrawing the process catalyst from the dehydrogenation process, modifying the process catalyst to form a modified catalyst, and adding the modified catalyst back to the dehydrogenation process. Where the process catalyst comprises from 0.1 wt. % to 10 wt. % of one or more metals chosen from gallium, indium, thallium, or combinations thereof, from 1 ppmw to 1000 ppmw of one or more metals chosen from platinum, palladium, rhodium, iridium, ruthenium, osmium, or combinations thereof, and at least 85 wt. % support. Where modifying the process catalyst comprises adding one or more of manganese, iron, chromium, vanadium, or aluminum.

A second aspect of the present disclosure may include the first aspect where the process catalyst comprises from 0.1 wt. % to 10 wt. % of gallium, from 1 ppmw to 1000 ppmw of platinum, and at least 85 wt. % support.

A third aspect of the present disclosure may include any of the previous aspects where the process catalyst is void of manganese and iron.

A fourth aspect of the present disclosure may include any of the previous aspects where the process catalyst comprises manganese, iron, or combinations thereof.

A fifth aspect of the present disclosure may include any of the previous aspects where modifying the process catalyst comprises adding manganese and where the modified catalyst comprises from 100 ppmw to 5000 ppmw of manganese.

A sixth aspect of the present disclosure may include any of the previous aspects where modifying the process catalyst comprises adding iron and where the modified catalyst comprises from 100 ppmw to 5000 ppmw of iron.

A seventh aspect of the present disclosure may include any of the previous aspects where modifying the process catalyst comprises adding chromium and where the modified catalyst comprises from 100 ppmw to 5000 ppmw of chromium.

An eighth aspect of the present disclosure may include any of the previous aspects where modifying the process catalyst comprises adding vanadium and where the modified catalyst comprises from 100 ppmw to 2000 ppmw of vanadium.

A ninth aspect of the present disclosure may include any of the previous aspects where modifying the process catalyst comprises adding aluminum and where the modified catalyst comprises from 0.5 wt. % to 10 wt. % of aluminum.

A tenth aspect of the present disclosure may include any of the previous aspects where modifying the process catalyst further comprises adding one or more of platinum or gallium.

An eleventh aspect of the present disclosure may include any of the previous aspects where modifying the process catalyst further comprises adding from 10 ppmw to 300 ppmw of platinum.

A twelfth aspect of the present disclosure may include any of the previous aspects where modifying the process catalyst further comprises adding from 0.1 wt. % to 0.5 wt. % of gallium.

A thirteenth aspect of the present disclosure may include any of the previous aspects where the support comprises one or more of alumina, silica, or combinations thereof.

A fourteenth aspect of the present disclosure may include any of the previous aspects where the withdrawing of the process catalyst occurs after the process catalyst has undergone at least 4 months of dehydrogenation process cycles.

A fifteenth aspect of the present disclosure may include any of the previous aspects where the process catalyst is withdrawn from the dehydrogenation process when there is a decrease in combustion activity of the process catalyst of at least 60%

It will be apparent to those skilled in the art that various modifications and variations can be made to the presently disclosed technology without departing from the spirit and scope of the technology. Since modifications combinations, sub-combinations and variations of the disclosed embodiments incorporating the spirit and substance of the presently disclosed technology may occur to persons skilled in the art, the technology should be construed to include everything within the scope of the appended claims and their equivalents. Additionally, although some aspects of the present disclosure may be identified herein as preferred or particularly advantageous, it is contemplated that the present disclosure is not limited to these aspects.

It is noted that the various details described in this disclosure should not be taken to imply that these details relate to elements that are essential components of the various embodiments described in this disclosure, even in cases where a particular element is illustrated in each of the drawings that accompany the present description. Unless specifically identified as such, no feature disclosed and described herein should be construed as “essential”. Contemplated embodiments of the present technology include those that include some or all of the features of the appended claims.

For the purposes of describing and defining the present disclosure it is noted that the term “about” are utilized in this disclosure to represent the inherent degree of uncertainty that may be attributed to any quantitative comparison, value, measurement, or other representation. The term “about” are also utilized in this disclosure to represent the degree by which a quantitative representation may vary from a stated reference without resulting in a change in the basic function of the subject matter at issue.

In relevant cases, where a composition is described as “comprising” one or more elements, embodiments of that composition “consisting of” or “consisting essentially of” those one or more elements is contemplated herein.

It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

It is noted that one or more of the following claims and the detailed description utilize the terms “where” or “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that any two quantitative values assigned to a property may constitute a range of that property, and all combinations of ranges formed from all stated quantitative values of a given property are contemplated in this disclosure. Where multiple ranges for a quantitative value are provided, these ranges may be combined to form a broader range, which is contemplated in the embodiments described herein.

1. A method for making light olefins by dehydrogenation, the method comprising:
operating a dehydrogenation process whereby a hydrocarbon-containing feed is converted to light olefins, wherein the dehydrogenation process utilizes a fluidized process catalyst that circulates between a reactor and a combustor, wherein the process catalyst comprises:
from 0.1 wt. % to 10 wt. % of one or more metals chosen from gallium, indium, thallium, or combinations thereof;
from 1 ppmw to 1000 ppmw of one or more metals chosen from platinum, palladium, rhodium, iridium, ruthenium, osmium, or combinations thereof;
and at least 85 wt. % support;
withdrawing the process catalyst from the dehydrogenation process;
modifying the process catalyst to form a modified catalyst, wherein the modifying of the process catalyst comprises adding one or more of manganese, iron, chromium, or vanadium; and
adding the modified catalyst back to the dehydrogenation process.

2. The method of claim 1, wherein the process catalyst comprises:
from 0.1 wt. % to 10 wt. % of gallium;
from 1 ppmw to 1000 ppmw of platinum;
and at least 85 wt. % support.

3. The method of claim 1, wherein the process catalyst is void of manganese and iron.

4. The method of claim 1, wherein the process catalyst comprises manganese, iron, or combinations thereof.

5. The method of claim 1, wherein the modifying of the process catalyst comprises adding manganese, and wherein the modified catalyst comprises from 100 ppmw to 5000 ppmw of manganese.

6. The method of claim 1, wherein the modifying of the process catalyst comprises adding iron, and wherein the modified catalyst comprises from 100 ppmw to 5000 ppmw of iron.

7. The method of claim 1, wherein the modifying of the process catalyst comprises adding chromium, and wherein the modified catalyst comprises from 100 ppmw to 5000 ppmw of chromium.

8. The method of claim 1, wherein the modifying of the process catalyst comprises adding vanadium, and wherein the modified catalyst comprises from 100 ppmw to 2000 ppmw of vanadium.

9. The method of claim 1, wherein modifying the process catalyst further comprises adding aluminum, wherein the modified catalyst comprises from 0.5 wt. % to 10 wt. % of aluminum.

10. The method of claim 1, wherein modifying the process catalyst further comprises adding one or more of platinum or gallium.

11. The method of claim 1, wherein modifying the process catalyst further comprises adding from 10 ppmw to 300 ppmw of platinum.

12. The method of claim 1, wherein modifying the process catalyst further comprises adding from 0.1 wt. % to 0.5 wt. % of gallium.

13. The method of claim 1, wherein the support comprises one or more of alumina, silica, or combinations thereof.

14. The method of claim 1, wherein the withdrawing of the process catalyst occurs after the process catalyst has undergone at least 4 months of dehydrogenation process cycles.

15. The method of claim 1, wherein the process catalyst is withdrawn from the dehydrogenation process when there is a decrease in combustion activity of the process catalyst of at least 60%.
